(19) United States
(12) Patent Application Publication
(10) Pub. No.: US 2018/0360881 A1
(43) Pub. Date: Dec. 20, 2018

(54) Compositions and Methods for Treatment of Her2 Positive Metastatic Breast Cancer

(71) Applicants: NANT HOLDINGS IP, LLC, Culver City, CA (US); NANTCELL, INC., Culver City 90232, CA (US); NANTKWEST, INC., Culver City, CA (US)

(72) Inventors: Shahrooz Rabizadeh, Culver City, CA (US); Patrick Soon-Shiong, Culver City, CA (US); Hans Klingemann, Culver City, CA (US)

(21) Appl. No.: 15/781,428

(22) PCT Filed: Dec. 9, 2016
(86) PCT No.: PCT/US16/66018

§ 371 Date: Jun. 4, 2018

Related U.S. Application Data
(60) Provisional application No. 62/265,382, filed on Dec. 9, 2015.

(51) Int. Cl. A61K 35/17; A61P 35/00; A61K 31/337; A61K 39/395; A61K 45/06; A61K 39/00

(52) U.S. Cl. CPC A61K 35/17; A61P 35/00; A61K 31/337; A61K 2039/5156; A61K 45/06; A61K 39/001106; A61K 39/3955

(57) ABSTRACT

Contemplated immunotherapies include co-administration of an activated NK cell that is further genetically modified and a cancer therapeutic agent. In preferred embodiments, activated NK cells are further modified to taNK cells, which include a chimeric antigen receptor (CAR) with affinity for a cancer specific antigen, a cancer associated antigen, or a tumor specific antigen. Activated NK cells can also be further genetically modified to include high affinity Fc receptor CD16a (V158). Appropriate cancer therapeutic agents include chemotherapeutic drugs (e.g., nant-paclitaxel) or cancer targeted antibodies (e.g., trastuzumab).

This application claims the benefit of priority to U.S. provisional application having Ser. No. 62/265,382, filed on Dec. 9, 2015.

### FIELD OF THE INVENTION

The field of the invention is pharmaceutically enhanced immunotherapy, especially as it relates to treatment with genetically modified natural killer cell that express a chimeric antigen receptor and microtubule inhibitors.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Tubulin-targeting drugs commonly produce in cell various defects in the mitotic spindle assembly, chromosome segregation, and cell division, and have therefore become an option in treatment of various cancers, and especially ovarian, breast, lung, bladder, prostate, esophageal, and other types of solid tumor cancers. Unfortunately, most of the tubulin-targeting drugs have serious side effects, particularly on fast dividing healthy cell. Moreover, delivery of at least some of these drugs (e.g., Paclitaxel) is limited due to poor solubility and protein binding. To overcome these difficulties, paclitaxel can be coupled to albumin nanoparticles. Nevertheless, toxicity still remains a significant problem.

Human epidermal growth factor receptor 2 (HER2) is over-expressed in up to 20% of invasive breast cancers and has been associated with more aggressive tumors, shorter relapse time, and poor overall survival. Significant clinical benefit has been achieved with HER2-targeted therapies, including monoclonal antibodies and small molecule inhibitors.

More recently, immunotherapy has become a promising option in cancer therapy and various approaches, typically based on T-cell and adenoviral gene delivery, have been reported at least moderate and temporary success. To increase cell-mediated killing in immunotherapy, natural killer cell (NK cell) can be employed. NK cell are an important effector cell type for adoptive cancer immunotherapy and early clinical trials in patients with advanced cancers have demonstrated the safety of unmodified and activated NK-92 cell (aNK), with no evidence of cytokine storm from multiple spaced infusions over several months. More recently, it was discovered that NK cell can be engineered to express one or more chimeric antigen receptors (CARs) to enhance their antitumor activity, and a stable clonal HER2-specific NK-92 cell line (HER2.taNK) mediated selective and sequential killing of HER2-expressing MDA-MB-453 cell in vitro (Mol Ther. 2015; 23(2):330-338). In vivo experiments also showed enrichment of such HER2.taNK cell in murine xenografts of MDA-MB-453/EGFP, and the HER2.taNK cell also reduced the number of pulmonary metastasis in a renal cell carcinoma model.

While such treatment appears to have substantially less toxicity and adverse side effects in a patient, complete and permanent remission or eradication of cancer cell is still not achieved in a consistent manner. Further, patients with advanced HER2-positive breast cancer frequently display primary resistance, and those who were initially sensitive to these therapies inevitably acquired resistance over time. Therefore, there is still a need for improved compositions and methods for NK cell based immunotherapy of cancers, as well as more effective therapies in HER-2 positive metastatic breast cancer.

### SUMMARY OF THE INVENTION

The inventive subject matter contemplates various pharmaceutical compositions, uses thereof, and methods for cancer immunotherapy in which an activated NK cell, or an activated NK cell having further genetic modification, is administered together with a cancer therapeutic agent to so help produce a more robust therapeutic response to a cancer or otherwise prevent relapse. In preferred aspects, the activated NK cell is immortalized or based on NK92 cells and the cancer therapeutic agent is an antibody (e.g. trastuzumab, etc) or a chemotherapeutic drug (e.g., paclitaxel, nant-paclitaxel, etc) administered metronomically at low dose over extended periods of time. In especially preferred embodiments, the activated NK cells are genetically modified to either express chimeric antigen receptors (CAR) against a cancer epitope or an Fc receptor (e.g., a CD16 receptor), more preferably a high affinity Fc receptor (e.g., a CD16a receptor, a CD16a receptor having valine at position 158, etc), and expression of an immune-stimulatory cytokine (e.g., IL-2), preferably intracellularly retained.

In some embodiments, a method of treating or preventing relapse of a cancer is contemplated by administering an activated NK cell, or an activated NK cell having further genetic modification, and co-administering a cancer therapeutic agent (e.g., antibody, chemotherapeutic drug, etc). In preferred embodiments, the chemotherapeutic drug is administered more than once before or after administering the activated NK cell. In such embodiments, the activated NK cell is preferably further modified to express a CAR against a cancer epitope.

It is preferred that NK cells of the inventive subject matter are modified to be activated, and in some embodiments are immortalized to facilitate propagation, and preferably are NK92 cells. It is contemplated that the activated NK cell can be further genetically modified to have reduced or abolished expression of a killer cell immunoglobulin-like receptor (KIR). Other contemplated modifications of the activated NK cell include enhanced antibody-dependent cell-mediated cytotoxic (ADCC) activity, expression of an Fc receptor (e.g., CD16) or high affinity Fc receptor (e.g., CD16a receptor, a CD16a receptor having valine at position 158, etc), or expression of an immune-stimulatory cytokine (e.g., IL-2), preferably intracellularly retained. The activated NK cells can be modified to express a chimeric antigen receptor with binding specificity for a cancer associated antigen, a cancer specific antigen, or a cancer neoepitope (e.g., via ectodomain with desired binding specificity, via scFv portion, etc). In some embodiments, it is advantageous to irradiate or otherwise effect the activated NK cells to reduce or avoid cell replication.

In some embodiments, the cancer therapeutic agents of the inventive subject matter include chemotherapeutic drugs (e.g., a tubulin targeting drug, paclitaxel, paclitaxel coupled to a protein, paclitaxel coupled to albumin nanoparticles, etc) and antibodies (e.g., antibody with binding specificity against a tumor associated antigen, a tumor specific antigen, or a cancer neoepitope, antibody with binding specificity for HER2, trastuzumab, etc).

Some compositions of the inventive subject matter include additional activated NK cells, some of which have been genetically modified to express a chimeric antigen receptor (e.g., with binding specificity for a cancer associated antigen, a cancer specific antigen, or a cancer neoepitope, etc), an Fc receptor (e.g., CD16 receptor, etc) or a high affinity Fc receptor (e.g., CD16a receptor, CD16a receptor having valine at position 158, etc), and expression of an immune-stimulatory cytokine (e.g., IL-2), preferably intracellularly retained, while other activated NK cells have not been genetically modified further.

In some methods of the inventive subject matter, administration of activated NK cells is followed by administration of additionally modified NK cells, or of NK cells that have not been genetically modified, in separate events (e.g., administration separated by at least one day, etc). In some embodiments, co-administration of a cancer therapeutic agent is performed before administering the activated NK cells, but it is contemplated that the cancer therapeutic agent can be administered after the activated NK cells, or both before and after. Preferably, chemotherapeutic drug s are administered using a low dose (e.g., 50%, 25%, or 10% of maximum approved dose for the drug when given alone, etc), preferably using a low dose repeatedly administered over one week or more, or administered at least two days apart. In preferred methods of treatment, dosing regimens of the cancer therapeutic agent and administration of activated NK cells is effective to reduce the size of a tumor in a patient.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a graph depicting an exemplary dosing schedule and comparative data for combined therapy according to the inventive subject matter.

FIG. 2A is a graph depicting post-treatment change in tumor volume for the treatments of FIG. 1.

FIG. 2B is a graph depicting post-treatment change in mean body weight for the treatments of FIG. 1.

FIG. 2C is a table depicting tumor growth inhibition for the treatments of FIG. 1 per measurement on day 32.

FIG. 3A is a graph depicting post-treatment change in tumor volume for a 90 day observation cycle.

FIG. 3B is a graph depicting post-treatment change in mean body weight for a 90 day observation cycle.

FIG. 4A is a table depicting dosing regimens for 10 sample groups.

FIG. 4B is a graph depicting post-treatment change in tumor volume for sample groups 1 through 6 of FIG. 4A.

FIG. 4C is a graph depicting post-treatment change in mean body weight for sample groups 1 through 6 of FIG. 4A.

FIG. 4D is a graph depicting post-treatment change in tumor volume for sample groups 1, 4, and 7 through 10 of FIG. 4A.

FIG. 4E is a graph depicting post-treatment change in mean body weight for sample groups 1, 4, and 7 through 10 of FIG. 4A.

FIG. 4F is a table depicting tumor growth inhibition and body weight change for each sample group of FIG. 4A

FIG. 5A is a depiction of IHC staining for cleaved caspase-3 for PBS treated cells in mice.

FIG. 5B is a depiction of IHC staining for cleaved caspase-3 for trastuzumab treated cells in mice.

FIG. 5CA is a depiction of IHC staining for cleaved caspase-3 for haNK Cells treated cells in mice.

FIG. 5D is a depiction of IHC staining for cleaved caspase-3 for combined haNK Cells and trastuzumab treated cells in mice.

### DETAILED DESCRIPTION

The inventors now have discovered that the therapeutic effect of administration of activated NK cells, or activated NK cells having further genetic modifications, can be synergistically increased by co-administration of a cancer therapeutic agent (e.g., antibody, chemotherapeutic drug, etc), in some embodiments in a metronomic low-dose regimen. In preferred aspects, the activated NK cells are further modified to express a chimeric antigen receptor (e.g., having binding specificity towards a cancer associated antigen, a cancer specific antigen, and a cancer neoepitope). The NK cells can also be modified to express a high affinity Fc receptor (e.g., a CD16a receptor, a CD16a receptor having valine at position 158, etc), and expression of an immune-stimulatory cytokine (e.g., IL-2), preferably intracellularly retained. In some embodiments, both activated NK cells having further genetic modification and activated NK cells having no further genetic modification are co-administered.

In particularly preferred aspects, the activated NK cells are NK-92 derivatives and are modified to have a reduced or abolished expression of at least one killer cell immunoglobulin-like receptor (KIR), which will render such cell constitutively activated (via lack of or reduced inhibition). Therefore, suitable modified cell may have one or more modified killer cell immunoglobulin-like receptors that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cell may also be commercially obtained from NantKwest (see URL www.nantkwest.com) as an aNK cell (activated natural killer cell). In still further contemplated aspects, the activated natural killer cell may also be derived from the patient and be activated ex vivo following known protocols in the art.

NK-92 is a cytolytic cancer cell line which was discovered in the blood of a subject suffering from a non-Hodgkins lymphoma and then immortalized ex vivo. NK-92 cells are derived from NK cells, but lack the major inhibitory receptors that are displayed by normal NK cells, while retaining the majority of the activating receptors. NK-92 cells do not attack normal cells nor do they elicit an unacceptable immune rejection response in humans. Characterization of the NK-92 cell line is disclosed in WO 1998/49268 and U.S. Patent Application Publication No. 2002-0068044.

In some embodiments aNKs are further genetically modified to express a chimeric antigen receptor. It is contemplated that such cells are based on immortalized or otherwise manipulated cell that allow rapid expansion to therapeutically relevant quantities. Thus, such cells may be genetically engineered to have extended replication potential, or be NK92 derivatives. In some embodiments, aNK cells are further genetically modified to express immune-stimulatory cytokine (e.g., IL-2), preferably intracellularly retained. In particularly preferred aspects, the further genetically modified aNKs comprise a recombinant nucleic acid that encodes a chimeric T-cell receptor. Most typically, the chimeric T-cell receptor will have an scFv portion or other ectodomain with binding specificity against a tumor associated antigen (e.g., CEA-CAM), a tumor specific antigen (e.g., HER2, PSA, PSMA, etc.), or a cancer neoepitope. There are numerous manners of further genetically engineering an aNK cell to express such chimeric T-cell receptor, and all manners are deemed suitable for use herein. For example, suitable chimeric antigen receptors may comprise an scFv portion and/or other ectodomain with binding specificity against the tumor associated antigen, tumor specific antigen, or cancer neoepitope. Alternatively, such cell may also be commercially obtained from NantKwest as a taNK cell (‘target-activated natural killer cell’).

Most typically, the chimeric antigen receptor is engineered to have affinity towards one or more cancer associated antigens (or carry an antibody with specificity towards the cancer associated antigen), and it is contemplated that all known cancer associated antigens are considered appropriate for use. For example, cancer associated antigens include CEA, MUC-1, CYPB1, etc. Likewise, where the chimeric antigen receptor is engineered to have affinity towards a cancer specific antigen (or carry an antibody with specificity towards the cancer associated antigen), it is contemplated that all known cancer specific antigens are considered appropriate for use. For example, cancer specific antigens include PSA, Her-2, PSA, brachyury, etc. Where the cells are engineered to have affinity towards a cancer neoepitope (or carry an antibody with specificity towards the cancer neoepitope), it is contemplated that all known manners of identifying neoepitopes will lead to suitable targets. For example, neoepitopes may be identified from a patient tumor in a first step by whole genome analysis of a tumor biopsy (or lymph biopsy or biopsy of a metastatic site) and matched normal tissue (i.e., non-diseased tissue from the same patient) via synchronous location guided alignment comparison (e.g., US2012/0059670) of the so obtained omics information. Additional filtering or identification of potential antigen or neoepitope targets can be based on expected or actual (non)expression level, subcellular location, or extracellular display of the potential targets. Identified neoepitopes can then be further filtered for a match to the patient's HLA type to increase likelihood of antigen presentation of the neoepitope. Most preferably, such matching can be done in silico. Antibodies against neoepitopes may also be isolated or generated as described in PCT/US14/29244.

Alternatively, or additionally, activated NK cell may also be combined with genetically modified NK cell that express a high-affinity Fcγ receptor (CD16), preferably where the receptor has bound an antibody that has binding specificity towards a cancer associated antigen, a cancer specific antigen, and a cancer neoepitope as noted above. In some embodiments, the NK cell is an NK-92 derivative modified to express the high-affinity Fcγ receptor (e.g., CD16, CD16a, CD16a with valine at position 158, etc). Sequences for high-affinity variants of the Fcγ receptor are well known in the art, and all manners of generating and expression are deemed suitable for use herein. Expression of such receptor is believed to allow specific targeting of tumor cells using antibodies produced by the patient in response to the treatment contemplated herein, or that are specific to a patient's tumor cells (e.g., neoepitopes), a particular tumor type (e.g., her2neu, PSA, PSMA, etc.), or that are associated with cancer (e.g., CEA-CAM). Advantageously, such cells may be commercially obtained from NantKwest as haNK cells (‘high-affinity natural killer cells) and may then be further modified.

Regardless of the type of activated NK cell and genetically modified NK cell it is contemplated that the cell are used in a pharmaceutical composition, typically formulated as a sterile injectable composition with between 10⁴-10¹¹ cell, and more typically 10⁵-10⁹ cell per dosage unit. However, alternative formulations are also deemed suitable for use herein, and all known routes and modes of administration are contemplated herein. In preferred embodiments, the cells are irradiated prior to administration in an effort to limit proliferation of the cells. It should be appreciated that other appropriate cellular modifications can be used to reduce or prevent cell proliferation. As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.).

While not limiting to the inventive subject matter, the ratio of activated NK cell to genetically modified NK cell is typically between 1:100 and 100:1, and more typically between 1:2 and 2:1, 1:3 and 3:1, 1:4 and 4:1, 1:5 and 5:1, 1:6 and 6:1, 1:7 and 7:1, 1:8 and 8:1, 1:9 and 9:1, 1:10 and 10:1, 1:20 and 20:1, 1:30 and 30:1, or 1:50 and 50:1. Moreover, it is contemplated that when administered over several injections (typically separated by at least one or two days), the ratio of activated NK cell to further genetically modified NK cell is typically the same. However, moderate changes in ratio are also expressly contemplated. For example, administration of the combined cell may be performed between once and five times (or more) in individual injections separated by one or two days. In another example, administration may comprise an initial one or two (or more) infusions of activated NK cell, which is then followed by subsequent (one or two or more) infusions of the further genetically modified NK cell. In still other examples, administration of the further genetically modified NK cell may alternate with administration of the activated NK cell.

While some embodiments may involve compositions or methods incorporating chemotherapeutic drugs, aNK cells, and taNK cells, it should be appreciated that such embodiments can substitute or add other cancer therapeutic agents or activated NK cells. For example, embodiments can include one or more chemotherapeutic drugs, one or more antibodies, aNK cells, taNK cells (having CARs with the same or different binding specificities), or haNK cells (having high affinity to the same or different binding substrates), or any combination of cancer therapeutic agents, aNK cells, taNK cells, and haNK cells.

It should be appreciated that while haNK cells and antibody (e.g. trastuzumab) treatments alone may significantly reduce tumor size, it is contemplated that combined treatments synergistically reduce tumor size or prevent relapse of a cancer. A potential mechanism for the synergy between cancer targeted antibody treatments and haNK cell treatments is antibody induced immunostimulation of the antibody targeted tumors for increased recognition and killing by haNK cells, likely amplified by the high-affinity CD16a receptor (V158) of the haNK cells. Thus, the inventive subject matter contemplates treatments for specific cancers by co-administering antibodies specifically targeted to a cancer, a tumor, or a cancer associated structure along with doses of NK cell platforms having high affinity for cancer cells in general (e.g., via NKG2D), or having high affinity for the specific cancer, tumor, or a structure associated with the specific cancer. Such treatments can further comprise chemotherapeutic drugs administered in a low dose, metronomic regimen to further enhance reduction of the tumor size of the cancer or prevention of relapse of the tumor.

Chemotherapeutic drugs of the inventive subject matter may be administered over an extended period (e.g., over at least one week, two weeks, three weeks, or more), and administration may thus be performed between two and 10 time, or between five and 15 time, or even more. Most typically, administration of the chemotherapeutic drug is separated by at least one or two days (in some cases even longer). Regardless of the number/interval of administration of the chemotherapeutic drug it is preferred that the chemotherapeutic drug is administered at a low dose. Especially preferred low doses are equal or less than 50%, or equal or less than 40%, or equal or less than 30%, or equal or less than 20%, or equal or less than 10% of the maximum approved dose for the drug when given alone. Low dose chemotherapy is thought to reduce adverse effects to the cells of the immune system, and as such helps preserve the patients immune reaction towards the cancer relevant antigens.

In some embodiments, the cancer therapeutic agent of the inventive subject matter is a chemotherapeutic drug. While not limiting to the inventive subject matter, it is generally preferred that the chemotherapeutic drug is an anti-microtubule agent, and most preferably paclitaxel (which may be coupled to a protein, e.g., albumin nanoparticles, such as in nant-paclitaxel, Abraxane®, etc). However, it should be appreciated that other drugs are also deemed suitable and preferred chemotherapeutic drugs include thalidomide, asparaginase, bevacizumab, 5-fluorouracil, hydroxyurea, streptozocin, 6-mercaptopurine, cyclophosphamide and various anti-metabolites (e.g., gemcitabine, etc), topoisomerase inhibitors, kinase inhibitors (e.g., receptor protein-tyrosine kinase inhibitors such as imatinib, sunitinib, etc), cytotoxic antibodies, platinum based drugs (e.g. cisplatin, etc), protease inhibitors (e.g., bortezomib, etc), antibiotics (e.g., bleomycin, doxorubicin, epirubicin, etc), and epipodophyllotoxins (e.g., etoposide, etc), etc. Most typically, however, it is preferred that these drugs will be administered in a metronomic low-dose regimen (see e.g., Cancer Treat Rev. 2014; 40(8):942-950).

In some embodiments the chemotherapeutic drug is coupled to a chimeric carrier protein conjugate, and especially a chimeric albumin drug conjugate. Most preferably, the chimeric carrier protein is an albumin that is genetically engineered to have one or more Fc binding domains, wherein the albumin is further coupled to the chemotherapeutic drug. Contemplated compositions will have the general structure of

T-x-A-y-D

in which T is a targeting moiety, x is a coupling mode of the targeting moiety with albumin or other carrier protein, A, and in which the chemotherapeutic drug, D, is coupled to the albumin or other carrier protein via coupling mode y. In most preferred aspects, the targeting moiety is an antibody or antibody derivative, x is an Fc binding protein, A is a human serum albumin, and D is a taxol derivative (e.g., paclitaxel) coupled to the albumin in an appropriate manner, and especially non-covalently.

Contemplated compositions of the chemotherapeutic drug may also omit the need to produce a chimeric carrier protein and rely on direct or indirect non-covalent binding of the targeting moiety and the therapeutic drug to the carrier protein. Such compounds will have the general structure of

T-n₁-A-n₂-D

in which T is a targeting moiety, n₁ is a non-covalent coupling mode of the targeting moiety with albumin or other carrier protein, A, and in which the chemotherapeutic drug, D, is coupled to the albumin or other carrier protein via non-covalent coupling mode n₂. In most preferred aspects, the targeting moiety is an antibody or antibody derivative, n₁ and n₂ are hydrophobic interaction, A is a human serum albumin, and D is a taxol derivative (e.g., paclitaxel) that is coupled to the albumin, and especially a taxol derivative (e.g., paclitaxel).

Depending on the particular chemotherapeutic drug and carrier protein, it should be appreciated that the manner of attachment may vary considerably, and suitable coupling between the carrier protein and the drug include non-covalent coupling, covalent coupling, and genetic fusion. For example, where the carrier protein is albumin, hydrophobic and/or non-covalent interaction with a drug may be employed. On the other hand, drugs may also be attached to the carrier via one or more specific chemical reactions, typically using a linkage to a free lysine on albumin, or via maleimide or DAC linkage to Cys 34 of albumin, etc. Therefore, and viewed from a different perspective, hydrophilic drugs may be covalently coupled to albumin, while hydrophobic drugs may be attached by hydrophobic interaction.

Some methods and compositions of the inventive subject matter include antibodies as a cancer therapeutic agent. It is generally preferred that such antibodies have a binding specificity toward a specific cancer, a tumor, or a cancer associated intracellular or extracellular structure. Especially preferred embodiments include antibodies with specific binding affinity to breast cancer tumors, more specifically HER2 positive breast cancer tumors (e.g., trastuzumab). However, it should be appreciated that the inventive subject matter contemplates use of other or additional cancer associated antibodies (e.g., alemtuzumab, abagovomab, abituzumab, adecatumumab, afutuzumab, alacizumab pegol, amatuximab, anatumomab mafenatox, anetumab ravtansine, apolizumab, ascrinvacumab, atezolizumab, bavituximab, belimumab, bevacizumab, bivatuzumab mertansine, brentuximab vedotin, brontictuzumab, catumaxomab, cetuximab, clivatuzumab tetraxetan, daratumumab, edrecolomab, ertumaxomab, etaracizumab ibritumomab tiuxetan, gemtuzumab ozogamicin, girentuximab, glembatumumab vedotin, ibritumomab tiuxetan, ipilimumab, labetuzumab, nivolumab, nimotuzumab, nivolumab, obinutuzumab, ofatumumab, oregovomab, panitumumab, pembrolizumab, pemtumomab, pertuzumab, ramucirumab, rituximab, tacatuzumab tetraxetan, tositumomab, trastuzumab emtansine, votumumab, zalutumumab, zanolimumab, etc).

In some embodiments the antibody is administered to a patient before a dose of activated NK cells. Doses of antibodies are preferably delivered to the patient at least three hours before administering the activated NK cells (e.g., NK92, aNKs, taNKs, haNKs, or combinations thereof), though it is contemplated that antibodies be delivered more than 4, 5, 6, 12, 18, 24, or 32 hours before NK cell treatment. Further, continuous or batch methods of administering the antibodies is, as well as the NK cells, are contemplated. Other cancer therapeutic agents (e.g. chemotherapeutic agents) may be administered as an alternative to antibodies, or in addition to them.

### Example 1

In one exemplary treatment method to determine efficacy of HER2.taNK in combination with metronomic Nant-paclitaxel (Abraxane) in a mouse model of HER2-positive breast cancer, the inventors used HER2.taNK cell as described previously (Mol Ther. 2015; 23(2):330-338). MDA-MB-453 cell (0.1 mL of 1×10⁸ cell/mL in 50% Matrigel) were injected s.c. into the left and right flank area of female NOD/SCID mice (7 to 8 weeks old). When tumors reached about 100 mm³, the mice were randomly assigned to 4 groups of 4 mice/group and dosed (i.v.) with saline, nant-paclitaxel, γ-irradiated (10 Gy) aNK cell/HER2.taNK cell, or nant-paclitaxel plus γ-irradiated (10 Gy) aNK cell/HER2.taNK cell (the cell were γ-irradiated to prevent the cell from replicating). Tumor growth was measured with calipers twice weekly prior to dosing, then twice weekly; animals were weighed before injection of cell, before dosing, then twice weekly. All data are presented as means±SEM, and the statistical analysis was done using ANOVA and Student's t-test.

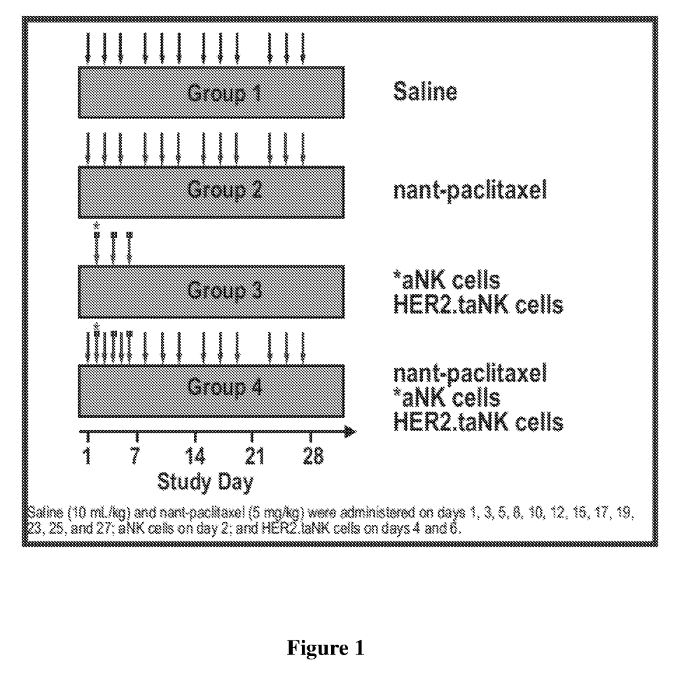

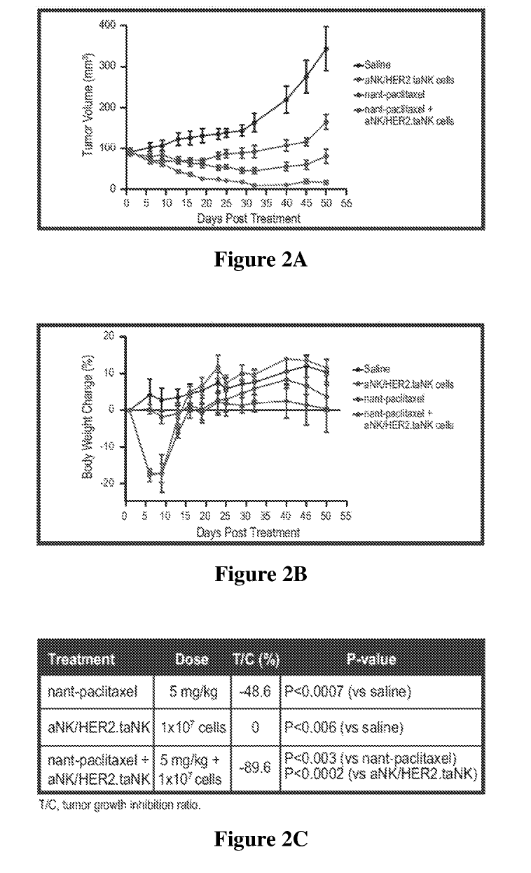

FIG. 1 schematically depicts four different treatment protocols of the described treatment method. Saline (10 mL/kg) and nant-paclitaxel (5 mg/kg) were administered on days 1, 3, 5, 8, 10, 12, 15, 17, 19, 23, 25, and 27; aNK cell on day 2; and HER2.taNK cell on days 4 and 6. As can be readily taken from FIGS. 2A and 2B, tumor volume increased with saline injection and was moderately suppressed using HER2.taNK cell and nant-paclitaxel individually. Unexpectedly, a combined treatment with aNK/HER2.taNK cell and nant-paclitaxel produced a significant and lasting reduction of tumor volume throughout the experimental period, while body weight was moderately and temporarily affected by HER2.taNK cell and aNK cell. The table in FIG. 2C reflects the changes expressed as tumor growth inhibition at day 32, and shows the substantial reduction in cancer growth using the combined treatment protocol.

It should be appreciated that while nant-paclitaxel alone and aNK cell/HER2.taNK cell alone significantly inhibited tumor growth in the mouse model of HER2-positive breast cancer, the combination of nant-paclitaxel plus aNK cell/HER2.taNK cell appeared to be synergistic, resulting in significant tumor regressions and significantly better efficacy vis-à-vis each agent alone. Notably, HER2.taNK cell were administered only twice early in the experiment, yet imparted a lasting impact on tumor growth. A potential mechanism for the synergy between low-dose paclitaxel and NK cell-based immunotherapy demonstrated in this set of experiments is paclitaxel-induced immunostimulation of tumors for increased recognition via antigen cascade and killing by the tumor-targeted NK-92 platform. Thus, results illustrate the potential for combining metronomic (low-dose) chemotherapy with NK-based immunotherapy in a clinical trial of patients with metastatic breast cancer and other cancers.

Therefore, contemplated treatments may include a cell based composition targeted to a specific cancer or cancer associated structure that is co-administered with a cancer therapeutic drug (e.g., chemotherapeutic drug) at low doses in a metronomic manner. Advantageously, such a treatment provides significant reduction of tumor size (or prevention of relapse of a cancer) with minimal adverse impact of the cancer therapeutic agent on the patient.

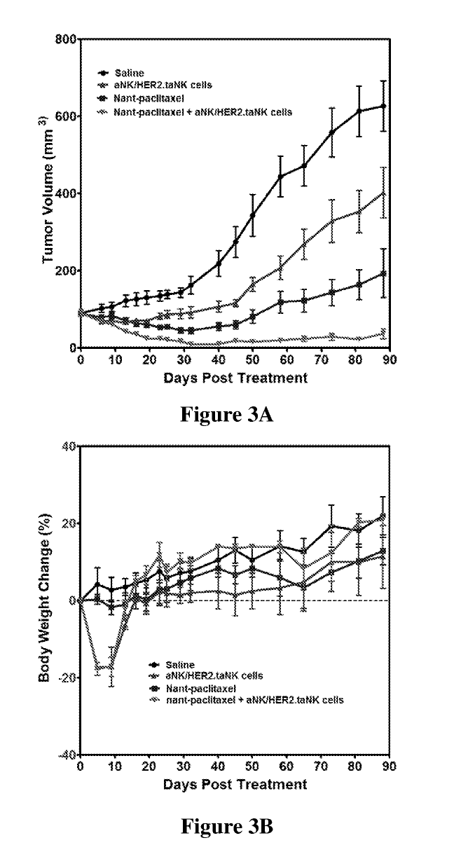

FIGS. 3A and 3B depict a 90 day treatment protocol and observation period similar to that described above. It should be noted in FIG. 3A that despite early (first 30 days) reduction of tumor volume under the separate aNK/HER2.taNK treatment regimen and the Nant-paclitaxel treatment regimen, the tumors continued to grow after the 30 day mark. Both treatment regimens failed to provide prolonged or maintained tumor reduction in the mouse model of HER2-positive breast cancer. Most notably, tumor volume under both treatment regimens surpassed the tumor volume when the treatments were initiated (i.e., 100 mm³). Indeed, approaching the 90 day mark the tumor volume of the aNK/HER2.taNK treatment regimen and the Nant-paclitaxel treatment regimen were ˜400 mm³ and −200 mm³, respectively.

Of particular note, the combined aNK/HER2.taNK and Nant-paclitaxel treatment regimen not only resulted in an unexpected synergistic reduction of tumor volume, but also unexpectedly resulted in prolonged and maintained tumor reduction in the mouse model of HER2-positive breast cancer. Indeed, while the separate aNK/HER2.taNK treatment regimen and the Nant-paclitaxel treatment regimen failed to prevent tumor growth as early as day 20 and day 40, respectively, the combined aNK/HER2.taNK and Nant-paclitaxel treatment regimen successfully both reduced tumor size and prevented significant tumor growth for the duration of the 90 day observation period. Such results suggest the combined aNK/HER2.taNK and Nant-paclitaxel treatment regimen is not only effective at synergistically reducing tumor size over a short period of time (30 days), but advantageously enhances an immune response to HER2 positive breast cancer or otherwise prevents renewed tumor growth for up to 90 days. It is particularly noteworthy that such evidence indicates treatments of the inventive subject matter are significantly effective to prevent relapse of a cancer.

Further, as shown in FIG. 3B, early body weight loss from the combined aNK/HER2.taNK and Nant-paclitaxel treatment regimen (first 10 days) quickly rebounded to surpass pretreatment body weight over the 90 day observation period, suggesting the apparent toxic effect of the combined treatment is short term or the patient otherwise acclimates to negative biological effects of the treatment regimen.

### Example 2

In a second exemplary treatment method to determine efficacy of haNK cells in combination with trastuzumab in a mouse model of HER2-positive breast cancer, the inventors used haNK cells as described previously. The haNK cells were developed by transfecting a parental aNK cell line with a bicistronic plasmid vector containing the high affinity V variant of CD16 (having valine at position 158) and an intracellularly-retained IL-2, which enables haNK cells to grow in the absence of exogenous IL-2. The plasmid contained some human origin sequences for CD16 and IL-2, neither of which have any transforming properties.

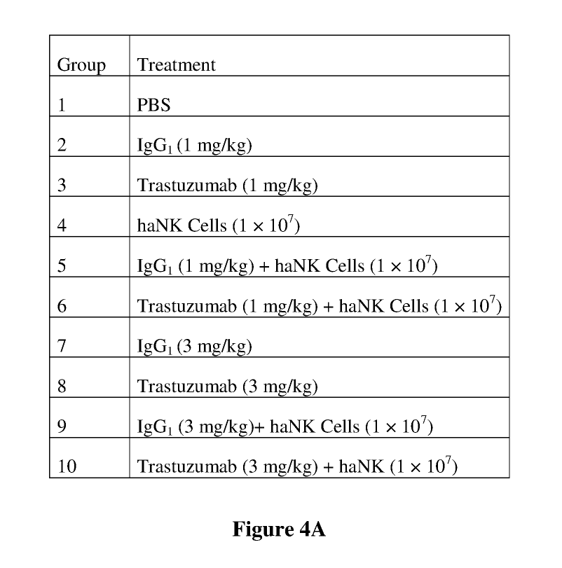

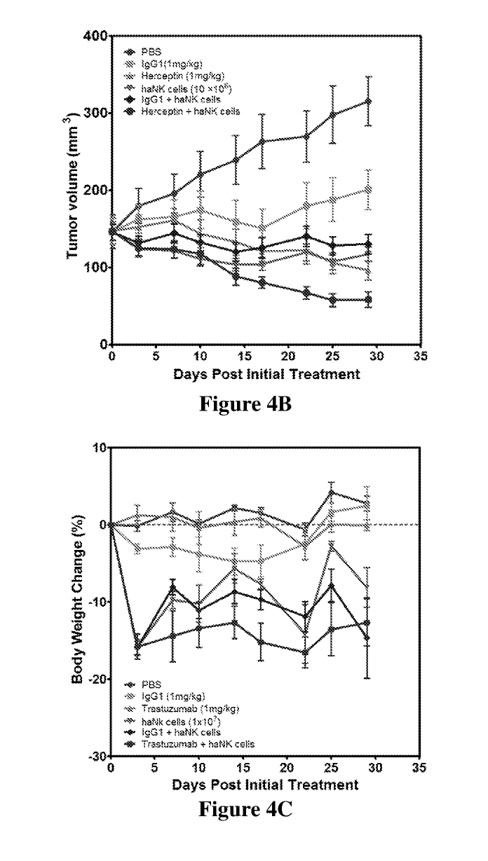

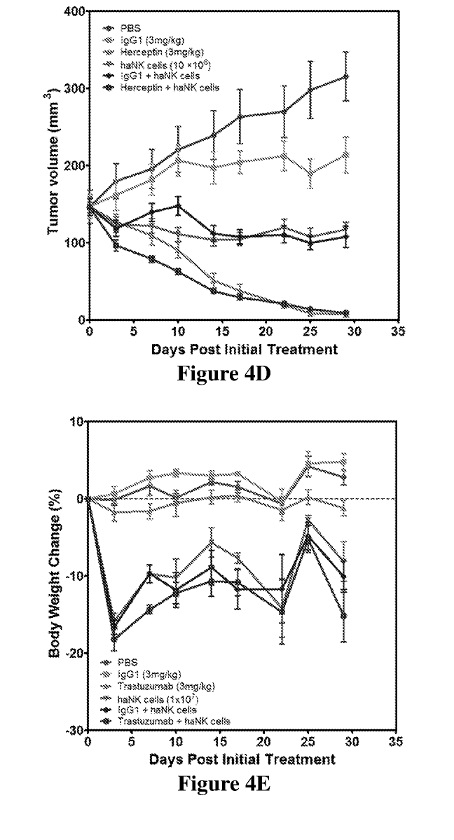

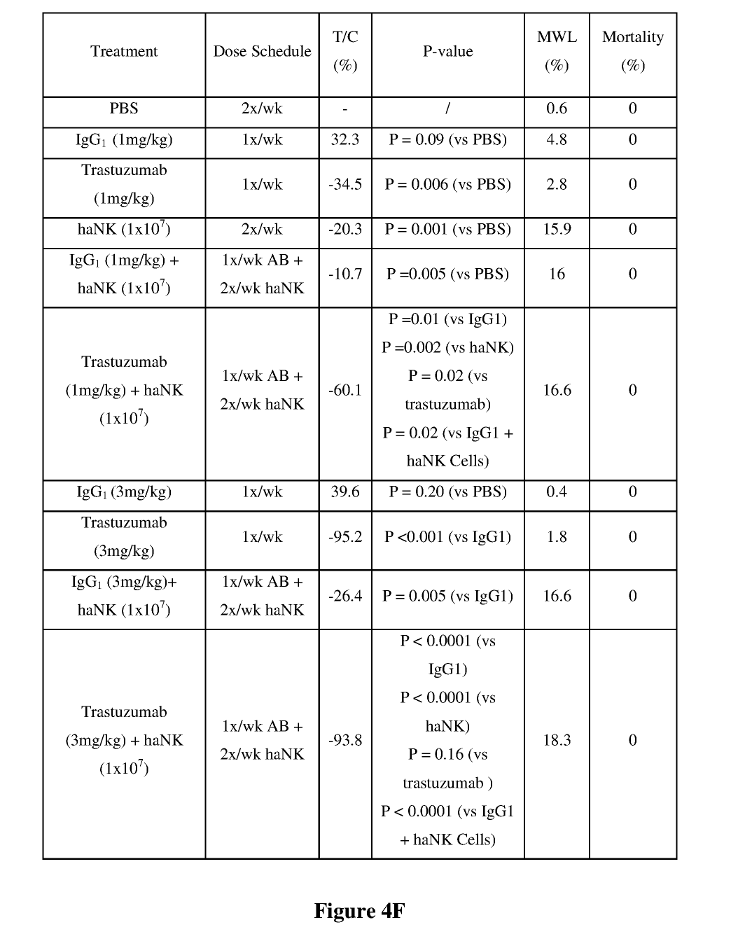

MDA-MB-453 cells (0.1 mL of 1×10⁸/mL in 50% Matrigel) were injected subcutaneously into the left and right flank area of female NOD-SCID IL2Rgamma^{null} (NSG, Jackson Laboratory) mice (7 to 8 weeks old). When tumors reached about 100 mm³, the mice were randomly assigned to one of ten groups of four mice/group as noted in FIG. 4A and dosed intravenously in the tail vein with saline (PBS), IgG₁ (in 1 gm/kg and 3 mg/kg doses), trastuzumab (in 1 gm/kg and 3 mg/kg doses), haNK Cells as described previously (1×10⁷ cells), combined IgG₁ and haNK Cells treatment (IgG₁ in 1 gm/kg and 3 mg/kg doses), and combined trastuzumab and haNK Cells treatment (trastuzumab in 1 gm/kg and 3 mg/kg doses). As depicted in FIG. 4F, the IgG₁ and trastuzumab control groups received doses once per week for four weeks, while the saline, haNK Cells, and combined IgG₁/trastuzumab and haNK Cells treatment regiments antibodies were administered once per week for four weeks and the haNK Cells were administered twice per week for four weeks. In the combined treatments, the mice received the antibody dose at least 3 hours prior to the injection of haNK cells.

Tumor growth and animal weights were measured twice weekly, as recorded in FIGS. 4B-4F. Statistical analyses of the difference in tumor volume or body weight change among the groups were evaluated using two-way ANOVA with repeated measures followed by the Bonferroni test, as reported in FIG. 4F (P-value). All the data were analyzed using GraphPad Prism software version 5. P<0.05 was considered to be statistically significant. The reported T/C (%) of FIG. 4F is ΔT/ΔC×100, where the ΔT and ΔC are the changes in the mean tumor volumes between day 29 and the first day of measurement for the treatment and control groups, respectively. The MWL is the maximum animal body weight loss over for the observed treatment cycle.

The recorded results for tumor size and body weight change for the control and 1 mg/kg dose antibody groups (groups 1-6 of FIG. 4A) are depicted in FIGS. 4B and 4C. It should be noted that saline and IgG₁ treatments each resulted in net-increases in tumor size, while trastuzumab (Herceptin®), haNK Cells, and combined IgG₁ and haNK Cells treatments resulted in a net-reduction in tumor size. The haNK cell treatment significantly inhibited tumor growth (T/C value of −20.3%), as did the trastuzumab significantly treatment (T/C values of −34.5% and −95.2% with 1 and 3 mg/kg, respectively).

Particularly of note, the combined treatment of trastuzumab (1 mg/kg) and haNK Cells surprisingly resulted in a synergistic and significant reduction in tumor size (T/C value of −60.1% per FIG. 4F). The significant difference between the reduction in tumor size from each of the separate trastuzumab regimen and haNK Cells regimen, and the synergistic reduction in tumor size of the combined trastuzumab and haNK Cells regimen suggests that dosing regimens of less than 1 mg/kg of trastuzumab, in combination with haNK Cells, could synergistically reduce tumor size of HER2 positive breast cancer models (e.g., no more than 0.9 mg, 0.8 mg, 0.7 mg, 0.6 mg, 0.5 mg, 0.4 mg, 0.3 mg, 0.2 mg, or 0.1 mg per kg).

The recorded results for tumor size and body weight change for the control and 3 mg/kg dose antibody groups (groups 1, 4, 7-10 of FIG. 4A) are depicted in FIGS. 4D and 4E. It should be noted that the IgG₁ 3 mg/kg and haNK cell treatment unexpectedly resulted in a more than doubled reduction in tumor size compared to the IgG₁ 1 mg/kg and haNK cell treatment (T/C value of −10.7% compared to −26.4, respectively; FIG. 4F). This suggests that increased concentrations of antibodies in general may enhance haNK Cells anti-tumor activity with respect to HER2 positive breast cancer.

Also of note, the trastuzumab 3 mg/kg treatment alone and the combined trastuzumab 3 mg/kg and haNK Cells treatment resulted in approximately the same tumor reduction. This suggests such a high dose of trastuzumab masks the synergistic effect of combined trastuzumab and haNK Cell treatments. As such, a lower dose of trastuzumab (e.g., 2.5 mg/kg, 2 mg/kg, 1.5 mg/kg, 1.2 mg/kg, 0.8 mg/kg, 0.6 mg/kg, 0.4 mg/kg, etc) is likely more efficient for tumor reduction of HER2 positive breast cancer in combined treatments of trastuzumab and haNK Cells.

Further, the fluctuation and net-decrease in body weight associated with all treatments including haNK Cells at 1×10⁷ cells per dose suggests that lower doses of haNK Cells (e.g., 5×10⁶, 1×10⁶, 5×10⁵, 1×10⁵, 5×10⁴, 1×10⁴, etc) are more effective at reducing the potential toxic effect of the dose on a patient, and potentially increasing the synergistic reduction of tumor size resulting from combined trastuzumab and haNK Cell treatments. However, it should be noted that the observed body weight loss associated with haNK Cells or the combination treatments were considered significant.

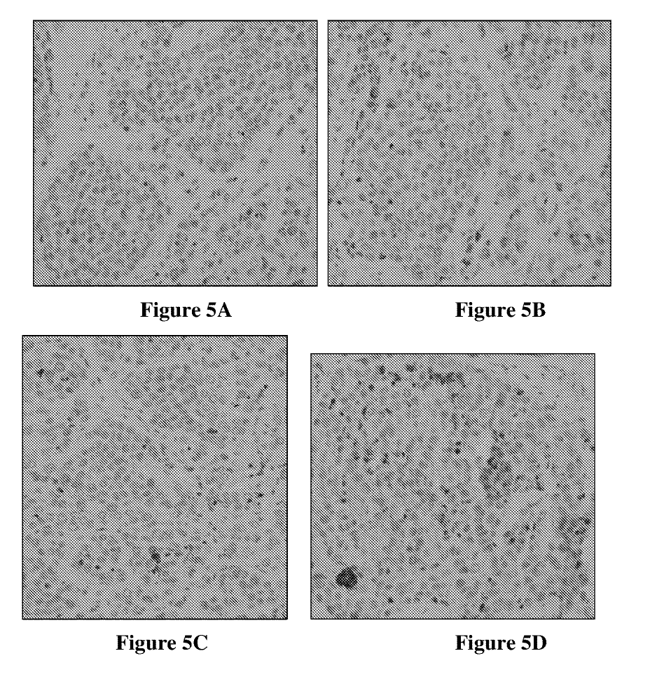

In addition, paraffin-embedded tumors were stained with cleaved caspase-3 rabbit monoclonal antibody (Cell Signaling, Cat #9579) at 1:100 and were counterstained with hematoxylin. As depicted in FIGS. 5A to 5D Such staining revealed increased caspase 3 activity associated with the combined trastuzumab (1 mg/kg) and haNK Cells treatment compared to the saline control and either agent alone.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1-26. (canceled)

27. A method of treating or preventing relapse of a cancer, comprising:
administering an activated NK cell, wherein the activated NK cell is further genetically modified; and
co-administering a cancer therapeutic agent selected from the group consisting of an antibody and a chemotherapeutic drug.

28. The method of claim 27 wherein the activated NK cell is further genetically modified to have a reduced or abolished expression of at least one killer cell immunoglobulin-like receptor (KIR).

29. The method of claim 27 wherein the activated NK cell is further genetically modified to enhance antibody-dependent cell-mediated cytotoxic activity.

30. The method of claim 27 wherein the activated NK cell is further genetically modified to express an Fc receptor.

31. The method of claim 30 wherein the Fc receptor is selected from the group consisting of a CD16 receptor, a CD16a receptor, and a CD16a receptor having valine at position 158.

32. The method of claim 27 wherein the activated NK cell is further genetically modified to express a chimeric antigen receptor having binding specificity towards a cancer associated antigen, a cancer specific antigen, or a cancer neoepitope.

33-35. (canceled)

36. The method of claim 32 wherein the chimeric antigen receptor has binding specificity towards HER2.

37. The method of claim 27 wherein the step of administering the activated NK cell further comprises administering a plurality of activated NK cells wherein a first portion of the activated NK cells are further genetically modified to express a chimeric antigen receptor having binding specificity towards a cancer associated antigen, a cancer specific antigen, or a cancer neoepitope.

38. The method of claim 37 wherein a second portion of the activated NK cells are further genetically modified to express an Fc receptor selected from the group consisting of a CD16 receptor, a CD16a receptor, and a CD16a receptor having valine at position 158.

39. The method of claim 37 wherein the step of administering the activated NK cell comprises administration of the activated NK cell followed by administration of the first portion of genetically modified activated NK cells in separate events, and wherein the separate events are spaced apart by at least one day.

40. The method of claim 27 wherein the step of administering the activated NK cell further comprises administering a second activated NK cell without further genetic modification.

41. The method of claim 40 wherein the step of administering the activated NK cell comprises administration of the second activated NK cell followed by administration of the genetically modified activated NK cells in separate events, and wherein the separate events are spaced apart by at least one day.

42. The method of claim 27 wherein the step of co-administering the cancer therapeutic agent is performed at least once before administering the activated NK cell.

43. The method of claim 27 wherein the step of co-administering the cancer therapeutic agent is performed at least once after administering the activated NK cell.

44. (canceled)

45. The method of claim 27 wherein the step of co-administering the chemotherapeutic drug is performed using a low dose.

46. (canceled)

47. The method of claim 45 wherein the low dose is less than 25% of a maximum approved dose for the drug when given alone.

48. The method of claim 45 wherein the low dose is less than 10% of a maximum approved dose for the drug when given alone.

49. The method of claim 45 wherein the low dose is repeatedly administered over at least one week.

50. The method of claim 51 wherein the low dose is administered at least two days apart.

51. The method of claim 44 wherein the chemotherapeutic drug is paclitaxel.

52. The method of claim 27 wherein the cancer therapeutic agent is an antibody having binding specificity towards HER2.

53. The method of claim 27 wherein the cancer therapeutic agent is trastuzumab.

54. The method of claim 27 wherein a patient's tumor size is reduced.

55-118. (canceled)
